(19) United States
(12) Patent Application Publication
(10) Pub. No.: US 2010/0318000 A1
(43) Pub. Date: Dec. 16, 2010

(54) SENSOR GUIDE WIRE

(75) Inventors: Pär Von Malmborg, Uppsala (SE); Leif Smith, Uppsala (SE)

Correspondence Address: FOLEY AND LARDNER LLP;SUITE 500, 3000 K STREET NW, WASHINGTON, DC, 20007, US

(73) Assignee: ST.JUDE MEDICAL SYSTEMS AB

(21) Appl. No.: 12/739,323

(22) PCT Filed: Oct. 24, 2008
(86) PCT No.: PCT/SE08/51208

§ 371 Date: Jul. 27, 2010

Related U.S. Application Data
(60) Provisional application No. 60/996,068, filed on Oct. 26, 2007.

(51) Int. Cl. A61M 25/09

(52) U.S. Cl. 600/585

(57) ABSTRACT

The present invention relates to a sensor guide wire for intravascular measurements of physiological variables in a living body, having a proximal shaft region, a flexible region and a distal sensor region. The sensor guide wire comprises, a sensor element provided in the sensor region, for measuring the physiological variable and to generate a sensor signal in response to said variable, a signal transmitting micro cable connected to the sensor element, and running along the sensor guide wire to the shaft region. The guide wire consists of, at least along the length of the flexible region, a guide wire tube that encloses the signal transmitting micro cable.

### FIELD OF THE INVENTION

The present invention relates to a sensor guide wire for intravascular measurements of physiological variables in a living body according to the preamble of the independent claims.

### BACKGROUND OF THE INVENTION

In many medical procedures, various physiological conditions present within a body cavity need to be monitored. These physiological conditions are typically physical in nature—such as pressure, temperature, rate-of-fluid flow, and provide the physician or medical technician with critical information as to the status of a patient's condition.

One device that is widely used to monitor conditions is the blood pressure sensor. A blood pressure sensor senses the magnitude of a patient's blood pressure, and converts it into a representative electrical signal that is transmitted to the exterior of the patient. For most applications it is also required that the sensor is electrically energized.

Some means of signal and energy transmission is thus required, and most commonly extremely thin electrical cables are provided inside a guide wire, which itself is provided in the form of a tube, which often has an outer diameter in the order of 0.35 mm, and oftentimes is made of steel. In order to increase the bending strength of the tubular guide wire, a core wire is positioned inside the tube. The mentioned electrical leads are positioned in the space between the inner lumen wall and the core wire.

A large flexibility of the sensor guide is advantageous in that it allows the sensor guide to be introduced into small and tortuous vessels. It should, however, also be recognized that if the core wire is too flexible, it would be difficult to push the sensor guide forward into the vessels, i.e. the sensor guide must possess a certain “pushability”. Furthermore, the sensor guide must be able to withstand the mechanical stress exerted on the core wire especially in sharp vessel bends.

Thus, the core wire must be carefully machined into different diameters at different portions of the guide wire, to provide the desired mechanical properties. And for a guide wire mounted sensor extra machining or wire forming of the core wire is usually necessary at the site where the sensor chip is placed.

A guide wire comprising a core wire provided with core wire portions of different diameters is disclosed in EP1475036 A1, assigned to the same assignee as in the present application.

The machining of the core wire is a time-consuming and thus expensive procedure, and one object of the present invention is to achieve a sensor guide wire that is easily manufactured.

U.S. Pat. No. 7,011,636 B2, also assigned to the same assignee as in the present application, discloses a guide wire provided with a central lumen filled with a core of electrically conductive material and which has an essentially constant diameter over its entire length. However, a drawback with this type of guide wire is that there is small possibilities to vary the mechanical properties over the length of the guide wire.

The object of the present invention is to achieve an improved sensor guide wire that obviates or reduces the above drawbacks.

### SUMMARY OF THE INVENTION

The above-mentioned object is achieved by the present invention according to the independent claim.

Preferred embodiments are set forth in the dependent claims.

The object of the present invention is thus to provide a sensor guide wire with the necessary stiffness, i.e. that has the required “pushability” to be introduced into small and tortuous vessels and that is less expensive to manufacture than presently used guide wires.

Another object is to provide a sensor guide wire with improved “torquability” (torque etc.) with in practise rotational symmetry and a minimum of whipping.

These objects of the present invention are achieved by a core wire free sensor guide wire.

The sensor guide wire for intravascular measurements of physiological variables in a living body, in accordance with the present invention, has a proximal shaft region, a flexible region and a distal sensor region, and comprises, a sensor element provided in the sensor region, for measuring the physiological variable and to generate a sensor signal in response to said variable, a signal transmitting micro cable connected to the sensor element, and running along the sensor guide wire to the shaft region. The guide wire consists of, at least along the length of the flexible region, a guide wire tube that encloses the signal transmitting micro cable.

The guide wire tube has the advantage of providing the sensor guide wire with the desired mechanical properties, such as flexibility and stiffness and column strength.

### SHORT DESCRIPTION OF THE APPENDED DRAWINGS

FIG. 1a shows a sensor guide wire according to the present invention.

FIG. 1b shows a cross-section A-A of the sensor guide wire shown in FIG. 1a.

FIG. 2 shows a tip region of a sensor guide wire provided with a tip wire according to the present invention.

FIG. 3 shows a self-locking tip wire according to the present invention.

FIG. 4 shows a sensor guide wire provided with a guide wire tube having helical grooves.

FIG. 5 shows a cross-section C-C in the sensor region, of the embodiment of the guide wire shown in FIG. 4.

FIG. 6 shows a cross-section A-A of the embodiment of the guide wire shown in FIG. 4.

FIG. 7 shows a sensor guide wire provided with a guide wire tube extending along the shaft region, the flexible, region, and the sensor region.

Throughout the figures same reference signs designates the same, or essentially the same feature.

### DETAILED DESCRIPTION OF PREFERRED EMBODIMENTS OF THE INVENTION

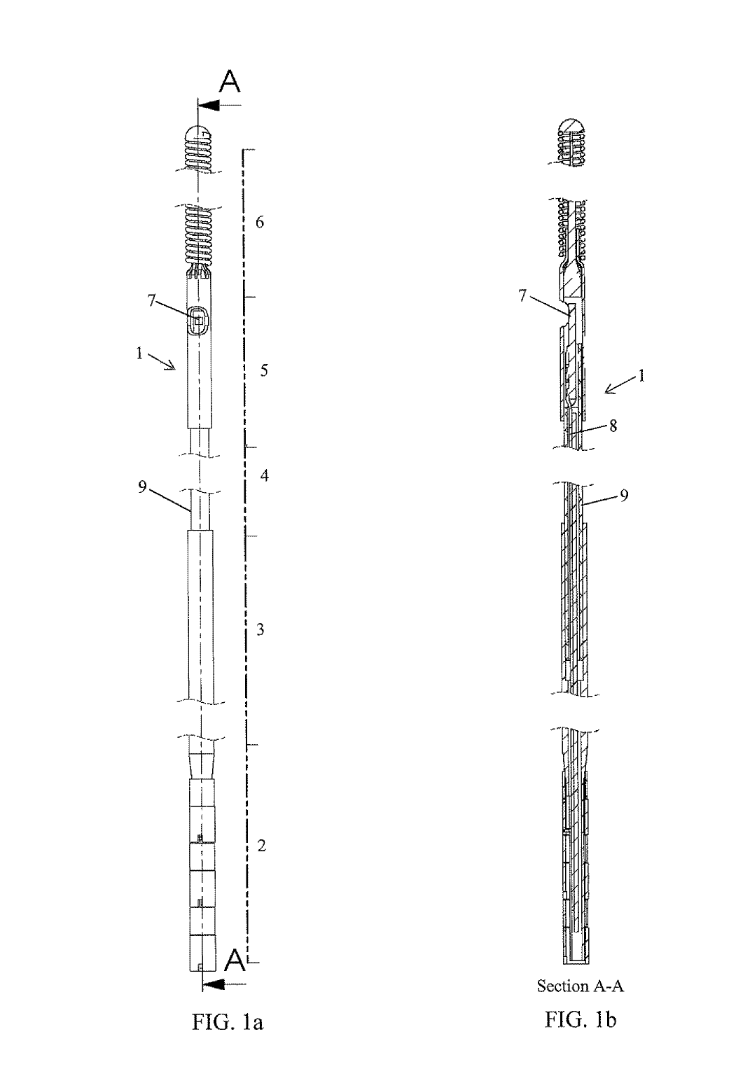

With references to the figures, and initially to FIGS. 1a and 1b, a sensor guide wire 1 for intravascular measurements of physiological variables in a living body, having a male connector region 2, a proximal shaft region 3, a flexible region 4, a distal sensor region 5, and a tip region 6, is disclosed. The sensor guide wire comprises, a sensor element 7 provided in the sensor region, for measuring the physiological variable and to generate a sensor signal in response to said variable, and a signal transmitting micro cable 8 connected to the sensor element 7, which is running along the sensor guide wire 1 to the shaft region 3.

The guide wire 1 consists of, at least along the length of the flexible region 4, a guide wire tube 9 that encloses the signal transmitting micro cable 8, as shown in FIG. 1b.

In a conventional sensor guide wire the signal transmitting cable is extending along the length of and next to the core wire, which means that the signal transmitting cable is not centered inside the sensor guide wire. A further advantage of enclosing the signal transmitting cable 8 with the guide wire tube 9, according to the present invention, as shown in FIG. 1b, is that the signal transmitting cable 8 may be symmetrically arranged within the guide wire tube 9, e.g. in the center, as no core wire exists.

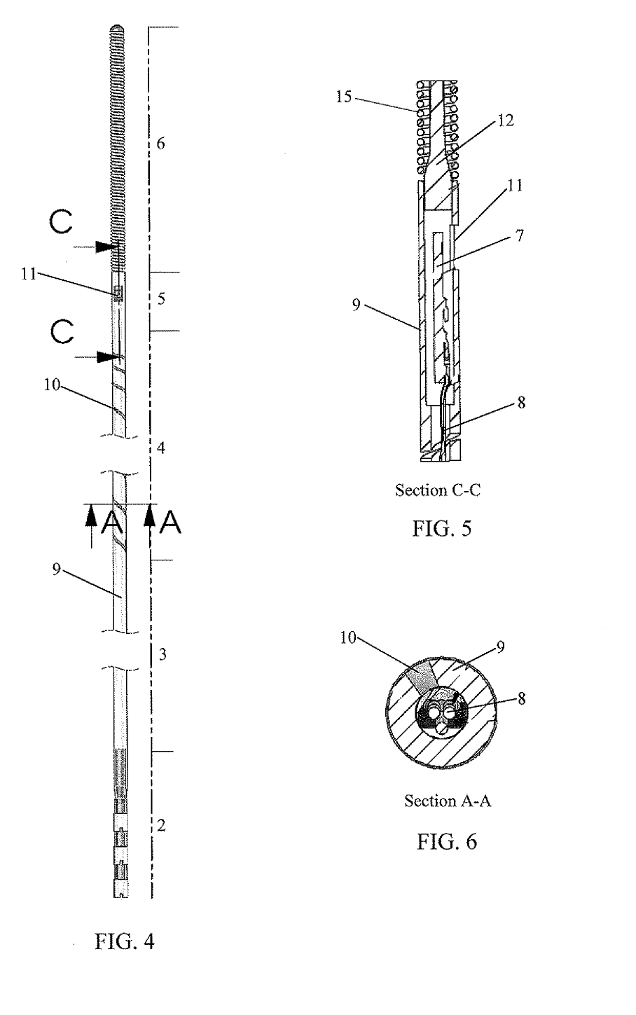

In another preferred embodiment, as illustrated in FIGS. 4-5 and 7, the guide wire tube 9 also extends along the sensor region 5 and encloses the sensor element 7 in the sensor region 5. This embodiment is advantageous since it reduces the number of components used in the sensor guide wire 1. In a conventional guide wire 1 a jacket is arranged to enclose the sensor element 7 in the sensor region 5. The preferred non-jacket embodiment of the present invention also has the advantage that it makes the sensor guide wire 1 easier to manufacture.

According to a preferred embodiment of the present invention, illustrated in FIGS. 2, 3 and 5, the tip region 6 of the guide wire 1 is provided with a tip wire 12 at least partly enclosed by a distal coil 15. The tip wire 12 provides the tip region 6 with the desired stiffness which is necessary to be able to push the sensor guide wire 1 forward into the vessels.

FIG. 2, shows a tip wire 12, according to one preferred embodiment, wherein the tip wire 12 comprises a tip core wire 13 extending in the centre of the tip wire 12 and an outer tip material 16 enclosing the tip core wire 13 in at least part of the tip region 6 of the guide wire 1, preferably adjacent to the sensor region 5. The diameter of the tip wire 12, adjacent to the sensor region 5, is adapted to the diameter of the jacket 17, and the tip wire 12 is inserted a predetermined distance into the jacket 17 in order to fasten the tip wire 12 to the jacket 17. The outer tip material 16 is preferably a super elastic material which is bonded to the tip core wire (13), which is shapable.

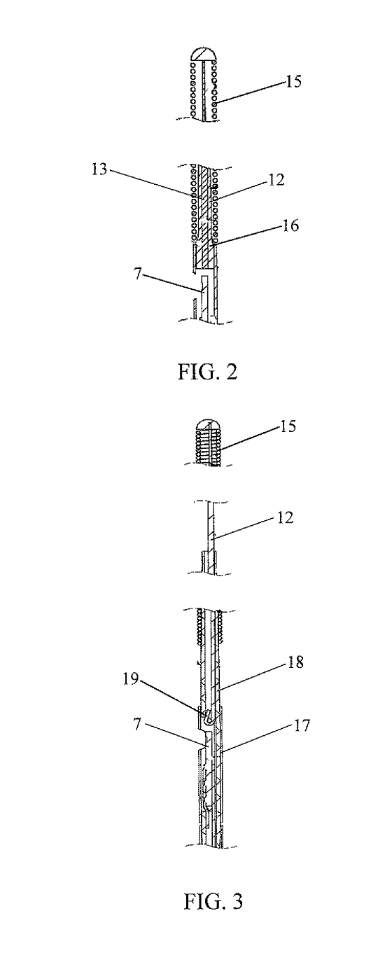

In FIG. 3, another preferred embodiment of the tip wire 12 is shown. According to this embodiment the tip wire 12 is provided with a self-locking feature in order to fasten the tip wire 12 to the jacket 17. A distal tube 18, extending along part of the tip region 6, is inserted a predetermined distance into the jacket 17. In order to fasten the tip wire 12 to the jacket 17, the tip wire 12 is provided with a shaped end 19. The shaped end 19 of the tip wire 12 is larger than the inner diameter of the part of the distal tube 18 inserted in the jacket 17, and the shaped end 19 of the tip wire 12 is thereby, and by means of a pull force, fastened inside the jacket 17.

In the preferred non-jacket embodiment, shown in FIGS. 4-5 and 7, the tip wire 12, is fastened to the guide wire tube 9 in a similar way as mentioned above.

In order to provide the guide wire 1 with the desired mechanical properties, e.g. the necessary stiffness and flexibility, the guide wire tube 9 can be made from various materials. In one preferred embodiment the guide wire tube 9 is divided into different lengths made from different materials (not shown), wherein the materials preferably are a super elastic alloy, such as Nitinol® (NiTi), and stainless steel. The lengths are joined together by means of any suitable technique, e.g. by welding, soldering, or gluing.

In another preferred embodiment of the present invention, as illustrated in FIG. 4, the guide wire tube's 9 outer side is provided with grooves 10. The grooves 10 are used to control the mechanical properties of the guide wire tube 9, and by varying for example the number of grooves 10 per length unit, the depth and/or the design of the grooves 10, the mechanical properties of the guide wire tube 9 will change.

According to the embodiment shown in FIG. 4, the grooves 10 are provided in the flexible region and arranged in a helical pattern. The stiffness of the guide wire tube 9 then depends on a pitch angle of the grooves 10, or in other words, the number of turns per mm. The higher the number of turns per mm is, the higher stiffness and conversely the lower the number of turns per mm is the higher flexibility. The number of turns per mm is 0, 5-50. Alternatively, the number of turns is varied along the length of, for example the flexible region 4, or the number of turns is set to be different in different regions.

In order to control the stiffness, the grooves 10 may, alternatively, have discontinuities in the groove windings. Other alternatives to grooves 10 arranged in a helical pattern, may be a plurality of grooves 10 of different or equal lengths extending in the longitudinal direction of the guide wire tube 9, preferably distributed all around the guide wire tube 9. The grooves 10 may also be arranged to extend crosswise to the longitudinal direction of the guide wire tube 9, or the grooves 10 may be arranged in any other way, suitable to control the stiffness of the guide wire tube 9.

The length of the guide wire tube 9 is varied in order to achieve different mechanical properties of the guide wire. The guide wire tube 9 is at least provided in the flexible region 4, alternatively the guide wire tube 9 is extending also along other adjacent regions, such as the shaft region 3, the sensor region 5, and the male connector region 2.

In a preferred embodiment of the present invention, as illustrated in FIGS. 4 and 7, the guide wire tube 9 extends along the length of the shaft region 3, the flexible region 4 and the sensor region 5. According to this embodiment the guide wire tube 9 is provided with an opening 11 for the sensor element 7.

FIG. 5 illustrates the preferred embodiment wherein the guide wire tube 9 extends along the length of the sensor region 5, and wherein an opening 11 for the sensor element 7 is provided in the guide wire tube 9. The signal transmitting cable 8 is connected to the sensor element 7 and is enclosed by the guide wire tube 9. FIG. 5 also shows a tip wire 12 fastened to the guide wire tube 9. The tip wire 12 is inserted a predetermined distance into the guide wire tube 9, in a similar way as described above in connection with FIG. 2, in order to fasten the tip wire 12 to the guide wire tube 9

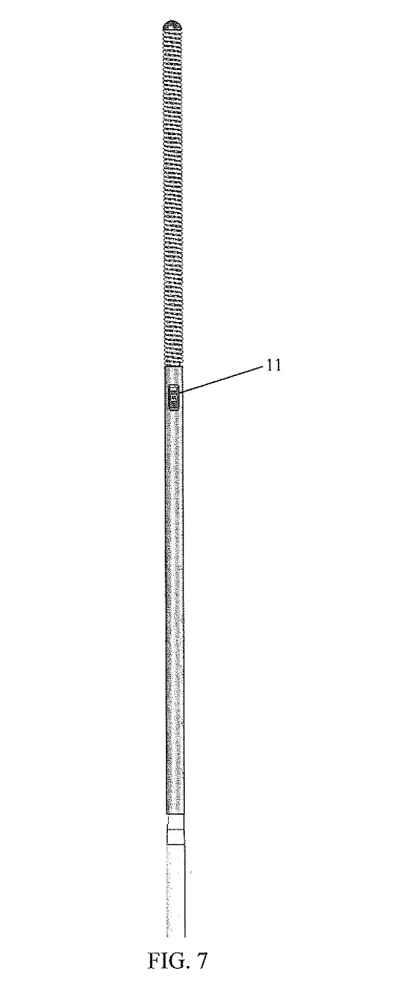

In a preferred embodiment of the present invention a plurality of signal transmitting cables 8 are enclosed by the guide wire tube 9, as illustrated in FIG. 6. FIG. 6 also shows a groove 10 arranged in the guide wire tube 9.

According to an alternative embodiment of the present invention, the guide wire tube 9 is provided with a coating at its outer surface.

The present invention is not limited to the above-described preferred embodiments. Various alternatives, modifications and equivalents may be used. Therefore, the above embodiments should not be taken as limiting the scope of the invention, which is defined by the appending claims.

1. Sensor guide wire for intravascular measurements of physiological variables in a living body, and having a proximal shaft region, a flexible region and a distal sensor region, the sensor guide wire comprises:
a sensor element provided in the sensor region, for measuring the physiological variable and to generate a sensor signal in response to said variable,
a signal transmitting micro cable connected to the sensor element, and running along the sensor guide wire to the shaft region,
characterized in that
the guide wire consists of, at least along the length of the flexible region, a guide wire tube that encloses the signal transmitting micro cable.

2. Sensor guide wire according to claim 1, wherein the guide wire tube also encloses the sensor element in the sensor region.

3. Sensor guide wire according to claim 1, wherein the guide wire tube is divided into different lengths made from different materials.

4. Sensor guide wire according to claim 3, wherein said materials are Nitinol (NiTi) and stainless steel.

5. Sensor guide wire according to claim 1, wherein the guide wire tube is provided with grooves at its outer surface.

6. Sensor guide wire according to claim 5, wherein the grooves are arranged in a helical pattern.

7. Sensor guide wire according to claim 5, wherein the grooves are provided in the flexible region.

8. Sensor guide wire according to claim 5, wherein the grooves have discontinuities in the longitudinal direction.

9. Sensor guide wire according to claim 5, wherein stiffness of the guide wire tube depends on the number of turns per mm of the grooves.

10. Sensor guide wire according to claim 9, wherein the number of turns per mm of the grooves is varying.

11. Sensor guide wire according to claim 9, wherein the number of turns per mm is 0, 5-50.

12. Sensor guide wire according to claim 1, wherein the guide wire tube extends along the length of the sensor region and wherein the guide wire tube is provided with an opening for the sensor element.

13. Sensor guide wire according to claim 1, wherein the guide wire tube extends at least partially along the length of the shaft region.

14. Sensor guide wire according to claim 1, wherein the guide wire is provided with a tip wire extending substantially along the tip region.

15. Sensor guide wire according to claim 14, wherein the tip wire comprises a tip core wire extending in the centre of the tip wire and an outer tip material enclosing the tip core wire in at least part of the tip region, wherein the outer tip material, preferably is a super elastic material which is bonded to the tip core wire.
